(19) United States
(12) Patent Application Publication
(10) Pub. No.: US 2024/0024061 A1
(43) Pub. Date: Jan. 25, 2024

(54) MEDICAL APPARATUS

(71) Applicant: SCOTT AIRWAY MANAGEMENT PTY LTD., Queensland (AU)

(72) Inventor: Paul Scott, Brisbane (AU)

(73) Assignee: SCOTT AIRWAY MANAGEMENT PTY LTD., Queensland (AU)

(21) Appl. No.: 18/040,736

(22) PCT Filed: Aug. 3, 2021
(86) PCT No.: PCT/AU2021/050844

§ 371 Date: Feb. 6, 2023

(30) Foreign Application Priority Data
Aug. 6, 2020 (AU) 2020902762
Sep. 10, 2020 (AU) 2020903237
Nov. 16, 2020 (AU) 2020904224

(51) Int. Cl. A61B 90/00; A61M 16/04

(52) U.S. Cl. CPC A61B 90/05; A61M 16/0493; A61B 1/267

(57) ABSTRACT

A closure device for use in combination with an endoscopy bite block, the bite block including a front piece to fit over a patient's mouth which has a central opening communicating with a passage extending into the patient's mouth to receive and pass an endoscopic tube, the closure device comprising a closure member configured for being inserted and retained in the passage during use wherein the closure comprises a flexible member extending across the opening, the flexible member further comprising a plurality of flexible segments being adapted to flex for accommodating an endoscopic tube to penetrate and pass through into the passage in an in-use engagement configuration wherein a portion of one or more of the flexible segments engages with outer walls of the tube to prevent expulsion of any droplets out of the patient's mouth during use.

### TECHNICAL FIELD

The present invention relates to medical apparatus particularly airway devices including but not limited to bite blocks for endoscopic use, adapted to provide for optimum subject comfort and tolerance, and especially such endoscopic bite blocks.

### BACKGROUND

Any references to methods, apparatus or documents of the prior art are not to be taken as constituting any evidence or admission that they formed, or form part of the common general knowledge.

There are many scenarios where medical apparatus is required to protect the patient's teeth whilst also maintain an airway in the patient. For example, a patient airway needs to be maintained when instruments, such as endoscopes, bronchoscopes transesophageal echocardiograms, are inserted through a person's mouth into the human body, in medical procedures. To prevent the patient from biting into the instrument, a bite block is used which is generally a plastic tube which is positioned in the patient's open mouth. The bite block can often be provided with oxygen supply ports which assist the patient in breathing and maintaining the airway whilst an endoscopic procedure is being carried out. The instrument is then inserted through the bite block opening into the stomach or other areas while the patient continues to breathe. The bite block opening is sufficiently sized (with a width that is larger than the diameter of the endoscope tube) to allow the endoscope tube to easily pass through and be maneuvered during use. One of the problems associated with the use of such bite blocks is that there is a gap left between the endoscope tube and walls of the bite block that define the opening. As a result, if a patient undergoing endoscopy, struggles to swallow the tube or has a gagging reflex by tensing their muscles or coughing due to stimulation of the vocal cords (due to secretions or surgical instruments touching the vocal cords) then infectious droplets may be expelled out of the gap (between the endoscope tube and the walls of the bite block).

In healthcare institutions, and public health settings, however, it is desirable not only to protect the patient from potentially harmful particulate matter, including biological pathogens but also to protect populations from a patient exhaling any pathogens into the environment where other healthcare providers may be present. It is therefore desirable to provide medical apparatus that can effectively protect the patient and surrounding healthcare providers from contacting any airborne viruses or bacterial that may otherwise spread during procedures such as endoscopy or providing breathing assistance due to involuntary coughing by the patient whilst they are being attended to by healthcare providers.

The use of oral protective devices is known and has been suggested in the past for protecting the teeth, tongue, and lips from biting injury during the administration of electroconvulsive therapy. These devices typically include a rear biting portion that fits between the teeth, an anterior rim that fits between the teeth and the lips, and a more anterior hollow tube that transmits air or oxygen into the oral cavity and thence to the lungs.

In healthcare institutions, and public health settings, however, it is desirable not only to protect the patient from potentially harmful particulate matter, including biological pathogens but also to protect populations from a patient exhaling such pathogens into the environment where other healthcare providers may be present. It is therefore desirable to provide medical apparatus that can effectively protect the patient and surrounding healthcare providers from contacting any airborne viruses or bacterial that may otherwise spread during procedures such as electroconvulsive therapy.

### SUMMARY OF INVENTION

In an aspect, the invention provides a medical apparatus for use in combination with an airway device to maintain a patient airway, the airway device comprising a proximal end and a distal end with a passage extending therebetween wherein the proximal end comprises an opening to receive a supply of gas for passage of air or medical gas and wherein the distal end is adapted to be inserted through the mouth of the patient, the medical apparatus comprising: a closure member configured for being inserted and retained in the passage during use wherein the closure comprises a flexible member extending across the opening, the flexible member further comprising a plurality of flexible segments being adapted to flex for accommodating an endoscopic tube to penetrate and pass through into the passage in an in-use engagement configuration wherein a portion of one or more of the flexible segments engages with outer walls of the tube to prevent expulsion of any droplets out of the patient's mouth during use.

In an embodiment, the closure member comprises a hollow closure body defining a lumen extending between a proximal end of the closure body and a distal end of the closure body with outer walls of the closure body being configured for engagement with inner walls defining the passage of the airway device.

In an embodiment, the flexible member is located at or adjacent one of the two ends of the closure body.

In an embodiment, the closure member comprises a plurality of said flexible segments being radially arranged relative to the opening.

In an embodiment, the one or more flexible segments are attached to a radially outer portion, preferably at one end, of the closure member.

In an embodiment, the plurality of flexible segments extending from a radially outer portion of the closure member in a radially inward direction towards the opening.

In an embodiment, the flexible segments are separated from each other by narrow cut-out sections formed continuously with the opening to allow the flexible segments to flex and accommodate an endoscopic tube in the opening, said endoscopic tube having a diameter that is greater than the width of the opening.

In an embodiment, the closure member comprises a profiled stop member to limit inward movement of the closure member into the passage.

In an embodiment, the stop member comprises a circumferential flange arranged circumferentially around the flexible segments.

In an embodiment, the circumferential flange has a diameter that is greater than the width of the lumen defined by outer walls of the closure body.

In an embodiment, the medical apparatus further comprises a flexible sheet adapted to be placed over a user's face wherein the flexible sheet comprises an opening dimensioned to receive outer walls of the closure body and prevent the proximal end of the closure body from being passed through the opening of the flexible sheet.

In an embodiment, the sheet is substantially impervious to air or liquids or one or more of the other medical gases.

In an embodiment, width of the opening of the sheet is dimensioned to be smaller than the width/diameter of the circumferential flange of the closure member to prevent the proximal end or of the closure member from passing through the opening of the mask and becoming uncoupled from the airway device or the closure member.

In an embodiment, the opening of the mask is dimensioned such that at least a portion of an underside surface of the circumferential flange of the closure member contacts a portion of the flexible sheet defining the opening to form a seal during use.

In an embodiment, the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

In an embodiment, the opening of the mask is located below the cut-out section for being positioned substantially above the mouth of the user.

In an embodiment, the medical apparatus further comprises a locking member configured to be received and engaged with a locking portion located on an outer wall portion of the passage extending between the proximal end and distal end of the airway device for detachably coupling said mask with the bite block.

In an embodiment, the locking member comprises a profiled projection member configured for engagement with an opening in the locking portion.

In an embodiment, during use, the projection member extends in a transverse direction relative to a longitudinal axis of the passage.

In an embodiment, the projection member extends in an in-use downwardly direction towards a radially central portion of the passage.

In an embodiment, the medical apparatus further comprises a passage engaging wall portion formed integrally with said flexible sheet, the wall portion being dimensioned for surrounding and engaging an outer wall of the passage, the passage engaging wall defining the opening at an end portion of the passage engaging wall.

In an embodiment, at least a portion of the flexible sheet, during use, engages with a back wall of a frontal portion of the airway device. Preferably, said at least portion of the flexible sheet is integrally formed with the passage engaging wall portion.

In another aspect, the invention provides a closure device for use in combination with an endoscopy bite block, the bite block including a front piece to fit over a patient's mouth which has a central opening communicating with a passage extending into the patient's mouth to receive and pass an endoscopic tube, the closure device comprising a closure member configured for being inserted and retained in the passage during use wherein the closure comprises a flexible member extending across the opening, the flexible member further comprising a plurality of flexible segments being adapted to flex for accommodating an endoscopic tube to penetrate and pass through into the passage in an in-use engagement configuration wherein a portion of one or more of the flexible segments engages with outer walls of the tube to prevent expulsion of any droplets out of the patient's mouth during use.

In an embodiment, the flexible sheet is attached or formed integrally with the outer wall of the anterior portion.

In an embodiment, during use, the closure device comprises flexible fastening tabs such that the opening of the airway device is flanked by said fastening tabs to allow the closure device to be fastened to the airway device.

In another aspect, the invention provides a medical apparatus for use in combination with an endoscopic mouthpiece, the endoscopic mouthpiece comprising: an annular bite portion for placement within the mouth of a patient so as to keep the mouth of the patient open and provide an open passage into the mouth of the patient with an opening to insert an endoscope therethrough; a frontal flange extending radially outwardly at one end of said annular bite portion for placement outside the mouth of the patient to overlap the patient's facial area surrounding the mouth of the patient; strap attachment members projecting upwardly from said flange; and one or more ports provided on said frontal flange for delivering gas to the patient for breathing; wherein the medical apparatus comprises a flexible sheet configured to be stretched and extended over the frontal flange of the mouthpiece to cover the opening of the annular bite portion, the flexible sheet comprising one or more slits for allowing insertion of an endoscope tube into the passage of the annular bite portion through the opening wherein the slit is formed by flexible segments being adapted to flex for accommodating the endoscopic tube to penetrate the flexible sheet and pass through into the passage in an in-use engagement configuration wherein a portion of one or more of the flexible segments engages with outer walls of the tube to prevent expulsion of any droplets out of the patient's mouth during use.

In an embodiment, the slit is located in between at least two spaced apart apertures dimensioned to be engaged with the strap attachment members and retain the flexible sheet in a stretched position to locate the slit across the opening of the annular bite portion.

In an embodiment, the flexible sheet further comprises one or more openings located to allow access into the one or more ports of the bite block when the flexible sheet is stretched and extended over the frontal flange of the mouthpiece to cover the opening of the annular bite portion.

In an embodiment, the medical apparatus comprises a first plurality of openings located on the flexible sheet to correspond with exhaling ports of the endoscopic mouthpiece to allow exhaled gas to exit the exhaling ports when the flexible sheet is stretched and extended over the frontal flange of the mouthpiece to cover the opening of the annular bite portion.

In an embodiment, the medical apparatus further comprises a second plurality of the openings to allow an inlet gas port of the endoscopic bite block to project outwardly from the endoscopic mouthpiece when the flexible sheet is stretched and extended over the frontal flange of the mouthpiece to cover the opening of the annular bite portion.

In an embodiment, a peripheral portion of the flexible sheet comprises a return lip for engaging an edge portion of the flange by lapping over the edge to maintain the sheet in the stretched position.

In an embodiment, the medical apparatus further comprises a flexible side wall extending towards the patient's face to define a hollow internal volume to accommodate the frontal flange wherein the return lip is located along a peripheral portion of the flexible side wall.

Preferably, the flexible side wall is integrally formed with the flexible sheet.

In an embodiment, the return lip is relatively thicker compared to the flexible sheet.

In an embodiment, the slit is substantially S-shaped formed by two flexible segments adapted to flex for accommodating the endoscopic tube to penetrate the flexible sheet and pass through into the passage in an in-use engagement configuration wherein respective portions of the two flexible segments engage with outer walls of the tube to prevent expulsion of any droplets out of the patient's mouth during use.

In an embodiment, at least two of the S-shaped slits are provided on the flexible sheet wherein the slits intersect each other.

In yet another aspect, the invention provides a medical apparatus for use in combination with an oral protector adapted for use for patients during electroconvulsive therapy (ECT), the oral device comprising a rear biting portion that fits between the teeth, an anterior portion that fits between the teeth and the lips, and a frontal portion configured to extend outwardly from the patient's mouth during use, the frontal portion further comprising an airway passage to transmit air or oxygen into the oral cavity and thence to the lungs of the patient using said oral device, the medical apparatus comprising: a flexible sheet adapted to be placed over a user's face wherein the flexible sheet comprises an attachment structure for attachment of the frontal portion such that in an attached configuration at least a part of the frontal portion extends forwardly from the mask to allow air or oxygen into the oral cavity and wherein the anterior portion and the rear biting portion extend rearwardly from the flexible sheet.

In an embodiment, the attachment structure comprises an opening that is sized to be narrower than a maximum length of the frontal portion to prevent the frontal portion from becoming uncoupled from the attachment structure during use in at least one operable configuration.

In an embodiment, the material of the sheet defining said opening is resilient to allow the opening to be stretched by a user into a stretched configuration to allow the frontal portion of the oral protector to be pushed through the opening.

In an embodiment, the opening is configured to receive and retain the anterior portion of the oral protector.

In an embodiment, the sheet is substantially impervious to air or liquids or one or more of the other medical gases.

In an embodiment, the frontal portion of the oral protector in combination with the opening are shaped to prevent the frontal portion from passing through the opening without stretching a portion of the flexible sheet surrounding the opening.

In an embodiment, the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

In an embodiment, the opening of the sheet is located below the cut-out section for being positioned substantially above the mouth of the user.

In an embodiment, an outer wall of the anterior portion defines a gap between the frontal portion and the rear biting portion for receiving the mask therein.

In an embodiment, a thickness of the flexible sheet is less than a width of the gap to allow the thickness of the flexible sheet to be accommodated in the gap.

In an embodiment, the flexible sheet is attached or formed integrally with the outer wall of the anterior portion.

In another aspect, the invention provides an oral protector in combination with a mask, the oral protector being adapted for use for patients during electroconvulsive therapy (ECT) wherein the oral protector comprises:
a rear biting portion that fits between the teeth,
an anterior portion that fits between the teeth and the lips, and
a frontal portion configured to extend outwardly from the patient's mouth during use, the frontal portion further comprising an airway passage to transmit air or oxygen into the oral cavity and thence to the lungs of the patient using said oral device; and
wherein the mask comprises:
   a flexible sheet adapted to be placed over a user's face wherein the flexible sheet comprises an attachment structure for attachment of the frontal portion such that in an attached configuration at least a part of the frontal portion extends forwardly from the mask to allow air or oxygen into the oral cavity and wherein the anterior portion and the rear biting portion extend rearwardly from the flexible sheet.

In an embodiment of the combination, the attachment structure of the mask comprises an opening that is sized to be narrower than a maximum length of the frontal portion of the oral protector to prevent the frontal portion from becoming uncoupled from the attachment structure during use in at least one operable configuration.

In an embodiment of the combination, the material of the sheet defining said opening is resilient to allow the opening to be stretched by a user into a stretched configuration to allow the frontal portion of the oral protector to be pushed through the opening.

In an embodiment of the combination, the opening is configured to receive and retain the anterior portion of the oral protector.

In an embodiment of the combination, the sheet is substantially impervious to air or liquids or one or more of the other medical gases.

In an embodiment of the combination, the frontal portion of the oral protector in combination with the opening are shaped to prevent the frontal portion from passing through the opening of the in an in-use configuration when a portion of the flexible sheet surrounding the opening is not held under a stretching force.

In an embodiment of the combination, on applying a stretching force to the portion of the flexible sheet surrounding the opening of the sheet, the frontal portion of the oral protector can be passed through the opening.

In an embodiment of the combination, the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

In an embodiment of the combination, the opening of the sheet is located below the cut-out section for being positioned substantially above the mouth of the user.

In an embodiment of the combination, an outer wall of the anterior portion defines a gap between the frontal portion and the rear biting portion for receiving the mask therein.

In an embodiment of the combination, thickness of the flexible sheet is less than a width of the gap to allow the thickness of the flexible sheet to be accommodated in the gap.

In an embodiment of the combination, the flexible sheet is attached or formed integrally with the outer wall of the anterior portion.

### BRIEF DESCRIPTION OF DRAWINGS

FIG. 1 is an exploded view of the medical apparatus 1000.

FIG. 2 is a frontal view of the medical apparatus 1000.

FIG. 3 is a top view of the medical apparatus 1000.

FIG. 3A is a rear perspective cut-away sectional view of the medical apparatus 1000.

FIG. 4 is a frontal view of the closure device 100.

FIG. 5 is a frontal perspective view of the closure device 100.

FIG. 6 is a top view of the closure device 100.

FIG. 7 is the side view of the closure device 100.

FIG. 8 is an in-use view of the closure device 100.

FIG. 9 is an in-use view of the medical apparatus 1000.

FIG. 10 is an exploded view of an endoscopic shield 200 in accordance with an embodiment.

FIG. 11 is a frontal perspective view of the endoscopic shield 200.

FIG. 12 is a rear perspective view of the endoscopic shield 200.

FIG. 13 is an in-use view of the endoscopic shield 200.

FIG. 14 is an exploded perspective view of a medical apparatus 2000 in accordance with an embodiment of the present invention.

FIG. 15 is a top view of the medical apparatus 2000 showing an uncoupled configuration wherein the mask 700 has been uncoupled from the oral protector 300.

FIG. 16 is a top view of the medical apparatus 2000 showing a coupled in-use configuration wherein the mask 700 is coupled to the oral protector 300.

FIG. 17 is a sectional view of the medical apparatus 2000 shown in FIG. 16.

FIG. 18 is a frontal view of the medical apparatus 2000.

FIG. 19 is a top perspective view of the oral protector 300.

FIG. 20 is a top view of the oral protector 300.

FIG. 21 is a frontal view of the oral protector 300.

FIG. 22 is a sectional view of the oral protector 300.

FIG. 23 is an exploded perspective view of another embodiment of a medical apparatus 1000′.

FIG. 24 is a side view of the medical apparatus 1000′.

FIG. 25 is a frontal perspective view of the medical apparatus 1000′.

FIG. 26 is a side view of the medical apparatus 1000′.

FIG. 27 is a frontal view of the medical apparatus 1000′.

FIG. 28 is a top view of the medical apparatus 1000′.

### DETAILED DESCRIPTION OF PREFERRED EMBODIMENTS

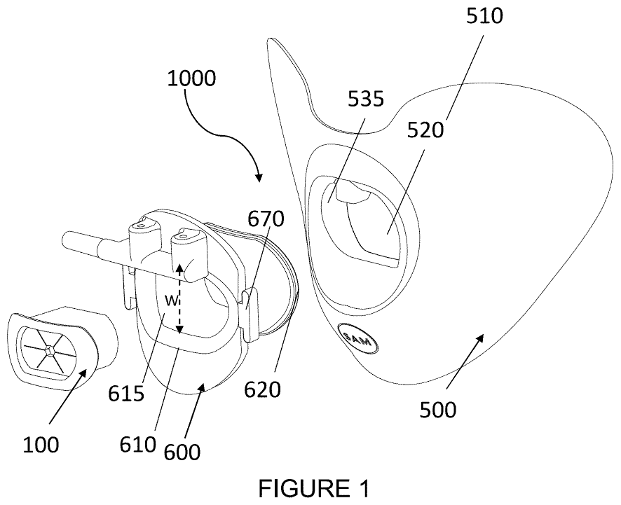

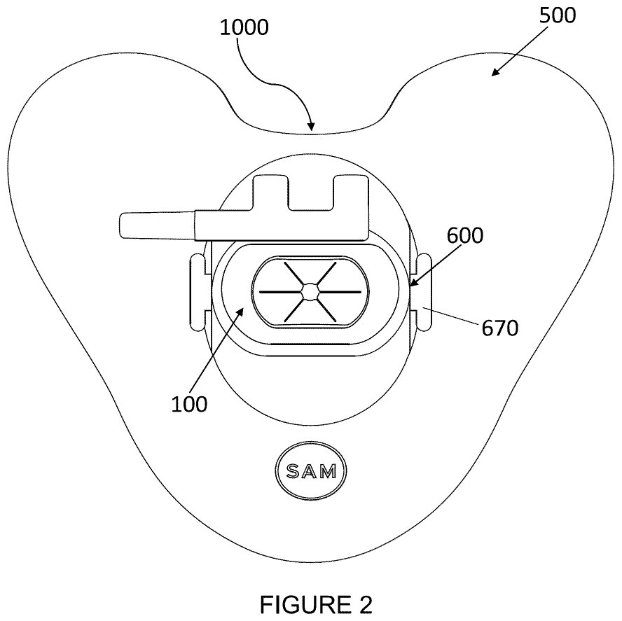

Referring to FIGS. 1 to 3, illustrate an embodiment of a medical apparatus 1000 that includes a closure device 100 for use in combination with an endoscopic bite block 600 and a mask 500. The closure device 100 and the mask 500 have been provided to shield healthcare providers from being accidentally exposed to infectious airborne droplets released by a patient while they have the endoscopic bite block 600 in their mouth.

For the purposes of describing the working of the medical apparatus 1000 it is important to note that the closure device 100 may be used in conjunction with the endoscopic bite block with or without the mask 500. Therefore, the use of the mask 500 is in no way essential for the working of the medical apparatus 100. The mask 500 has been described in further detail in the foregoing sections of the specification.

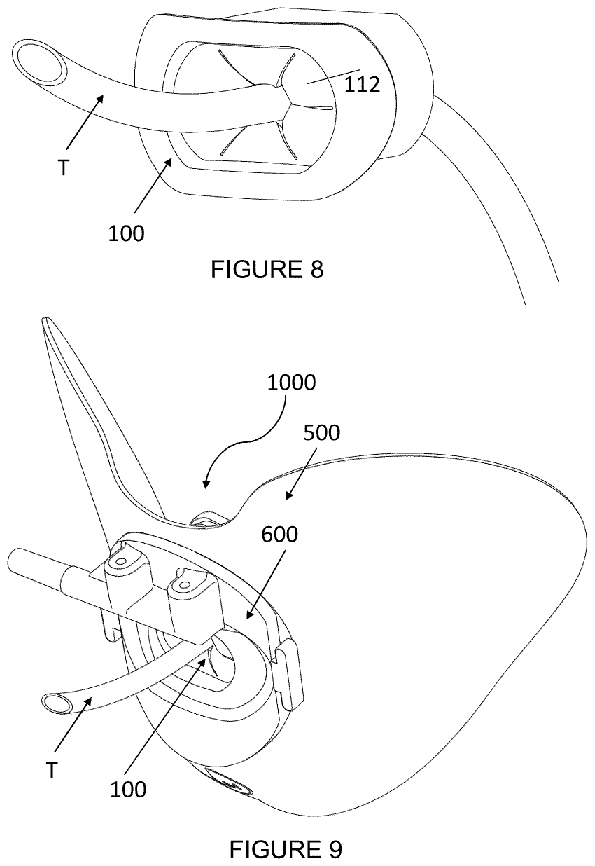

The exemplary bite block 600 shown in FIGS. 1 to 3 and 9 includes a front piece 650 to fit over a patient's mouth which has a central opening communicating with a passage 615 extending into the patient's mouth to receive and pass an endoscopic tube. The passage 615 that extends between the proximal end 610 (that receives the endoscope tube) and a distal end 620 (which is generally located inside the patient's mouth while endoscopic examination is underway. The passage 615 in the bite block 600 is defined by passage walls 616 and is sufficiently larger than the endoscopy tube T (shown in FIGS. 8 and 9) to be passed through and allow some residual volume around the endoscopy tube which allows air to pass through the passage 615 so that the patient can utilize that air for breathing while endoscopic examination is being carried out. The closure device 100 is configured for being inserted and retained in the passage 615 as shown in FIGS. 1 to 3. In an initial operable configuration, the closure device 100 can be plugged into the passage 615 before commencing endoscopic analysis and before inserting the endoscopic tube T into the passage 615.

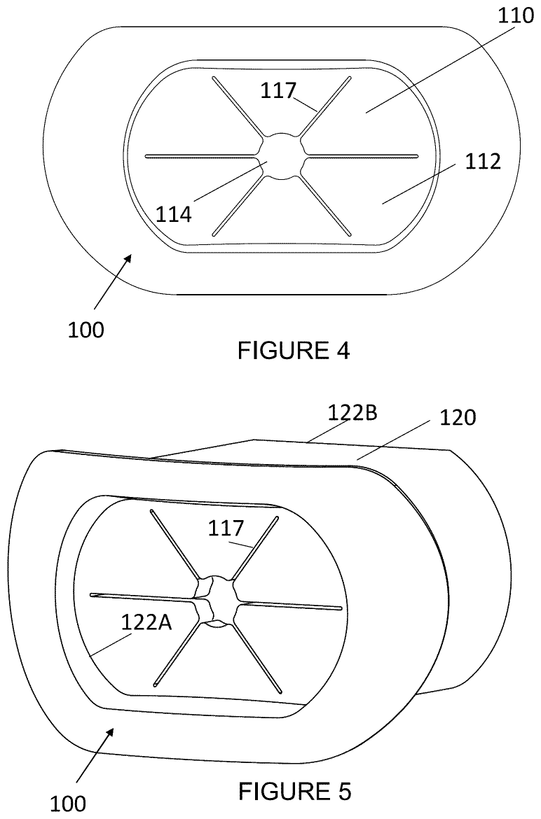

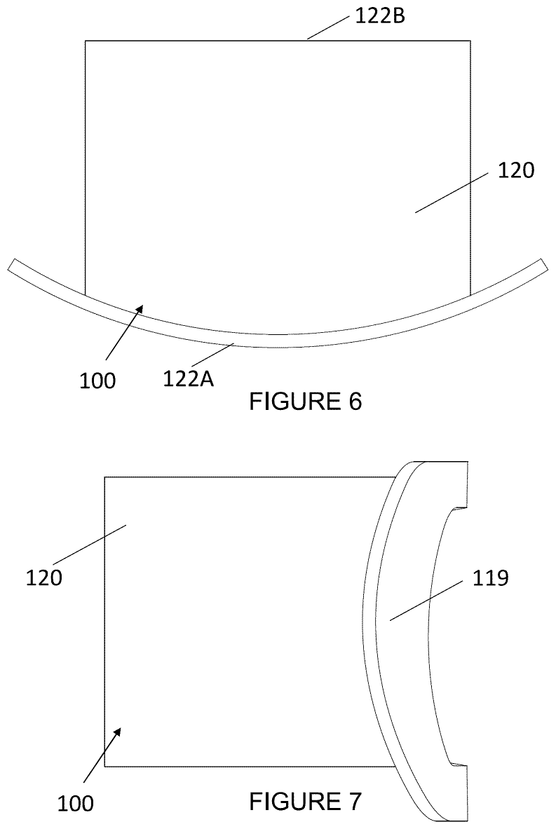

Referring to detailed views of the closure member 100 in FIGS. 4 to 8, the closure device 100 comprises a flexible member 110 with a plurality of generally triangular flexible segments 112. A centrally located aperture 114 is provided in the flexible member 110 and the width of the aperture is preferably smaller than the diameter of the endoscope tube T. As will be described in the foregoing sections, the flexible segments 112 are adapted to flex as the endoscope tube T is pushed into the aperture 114 for accommodating the endoscope tube T within the aperture 114.

The closure device 100 includes a hollow and substantially cylindrical closure body 120 defining a lumen 122 that extends between a proximal end 122A (that receives the endoscope tube T) and a distal end 122B which, during use, is pushed into the passage 615. The flexible segments 112 are radially arranged relative to the aperture 114 and each segment 112 is attached to a radially outer portion of the closure body 120 and extends towards the central aperture 114 in a radially inward direction. Each flexible segment 112 is separated from an adjacent segment by narrow cut-out sections 117 that are formed continuously with the central aperture 114. This configuration allows each of the flexible segments 112 to flex when the endoscope tube T is pushed into the aperture 114 and results in engagement between the flexible segments 112 and the outer walls of the endoscope tube T (See FIGS. 8 and 9) whilst also expanding the size of the aperture 114 to allow the endoscope tube T to pass through. The flexing of the flexible segments 112 during use prevents any large gaps between the endoscope tube T and the flexible segments during use. As a result, the flexible segments 112 function like a curtain and prevent expulsion of any droplets out of the patient's mouth during use whilst the endoscope T is in an inserted and in-use configuration.

The closure device 100 also includes a profiled stop member 119 to limit inward movement of the closure device 100 into the passage 615 of the bite block 600. In the preferred embodiment, the profiled stop member 119 comprises a circumferential flange that substantially surrounds the flexible segments 112 of the flexible member 110 and is relatively thicker and more rigid when compared with the flexible segments 112. Advantageously, the overall outer diameter of the profiled stop member 119 is greater than the width of the passage 615 of the bit block 600 in order to prevent the proximal end 122A from being accidentally pushed into the passage 615.

It is important to appreciate that in at least some embodiments, the closure device 100 may be integrally formed with the bite block 600 wherein the closure body 120 is integrally formed with the inner walls defining the passage 615 of the bite block 600.

As previously discussed, a mask 500 may be additionally used in conjunction with the bite block 600 and the closure device 100. The mask 500 is configured to be detachably coupled with the bite block 600 (as will be explained in further detail) and comprises a flexible sheet 510 that can be placed over the patient's face area surrounding the mouth of the patient. The flexible sheet 510 is sufficiently flexible to confirm to the contours of the patient's face. The flexible sheet 510 comprises an opening 520 that is located radially inwardly relative to the peripheral portions of the flexible sheet 510. The opening 520 is sized to allow the distal end 620 of the bite block 600 to pass through the opening 520. It is important to note that the opening 520 is dimensioned to be smaller than the width/diameter (denoted by W in FIG. 1) of a circumferential flange 640 located at the distal end 620 to prevent the distal end 620 from withdrawing through the opening 520 of the mask 500 and becoming uncoupled from the bit block 600.

The sheet 510 extends between two sides 532 and 534 with top and bottom portions 531 and 533 of the sheet 510 extending therebetween with the opening 520 being located substantially centrally relative to the peripheral portions of the sheet 510. The top portion 531 comprises a cut-out section 530 for receiving a nose of the user, the cut-out section 530 being flanked by a top left portion and a top right portion of the flexible sheet 510 to allow the cut-out section 530 to be positioned substantially above the mouth of the user when the mask 500 is coupled with the bite block 600 during use.

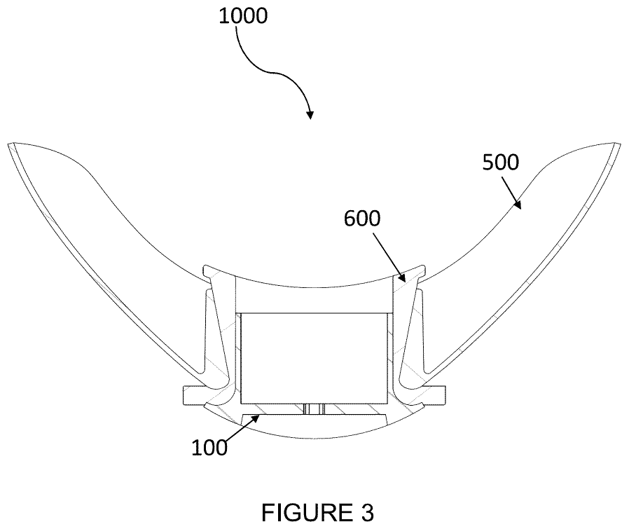

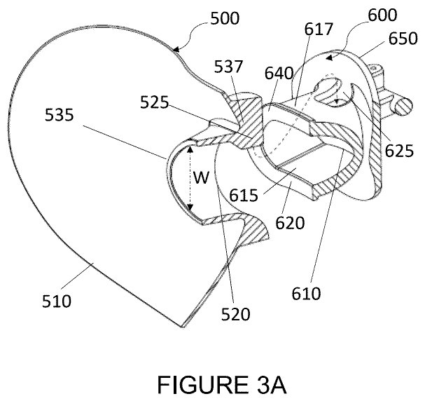

The mask 500 also includes a passage engaging portion 535 that is formed integrally with the flexible sheet 510 that is dimensioned to surround and engage an outer wall 617 of the passage 615. The passage engaging portion 535 extends from the opening 520 (that is located adjacent the proximal end 610) towards the distal end 620 of the passage 615. The passage engaging portion 535 comprises the wall(s) that define the opening 520 in the mask 500 within which the passage 615 of the bite block 600 is inserted. The passage engaging portion 535 of the mask 500 is also flexible and is dimensioned to snap onto the outer walls of the passage 615 which prevents the mask 500 from becoming decoupled from the bite block 600. Advantageously, thickness of the passage engaging portion 535 is slightly greater than thickness of the flexible sheet 510. The passage engaging portion 535 is formed continuously with a thickened turned portion 537 (See FIG. 3A) that is connected with the flexible sheet 510. A part of the flexible sheet portion 510 contacts a back face of the front piece 650 of the bite block 600 during use in the coupled configuration.

The mask 500 also includes a profiled and flexible locking member 525 that is configured to be received and engaged with a locking portion 625 located on the outer wall portion 617 of the passage 615 for detachably coupling said mask with the bite block. In the preferred embodiment, the locking member 525 extends downwardly in a transverse direction relative to a longitudinal axis of the passage provided in the bite block 600. The locking arrangement provided by the locking member 525 prevents the mask 500 from becoming decoupled from the bite block 500.

The front piece 650 for the bite block 600 also includes projecting holding members 670 that are maintained in a spaced relationship relative to an outer walls of the flexible sheet 510 to allow the user to hold the holding members 670 and pull the mask 500 away from the coupled bite block 600 to release the locking member 525 out of the locking portion 625.

In the coupled position, the flexible sheet 510 surrounds the front piece 650 and extends over the patient's face to surround the mouth region of the patient. As a result, any droplets inadvertently released by the patient (whilst endoscopy is being conducted) is captured along the inner walls of the flexible sheet 510 positioned in close proximity to the user's mouth. As a result, the mask 500 prevents the spread of such droplets and makes the working environment for the healthcare providers much safer and hygienic.

In some embodiments, the mask 500 may be integrally formed with the bite block 600. In such an embodiment, the flexible sheet 510 may be integrally with the front piece 650 of the bite block 600 and the locking arrangement as previously described may not be necessary. It would also be understood that one or ports may be provided in the bite block for additionally supplying oxygen or other gases into the mouth of the patient.

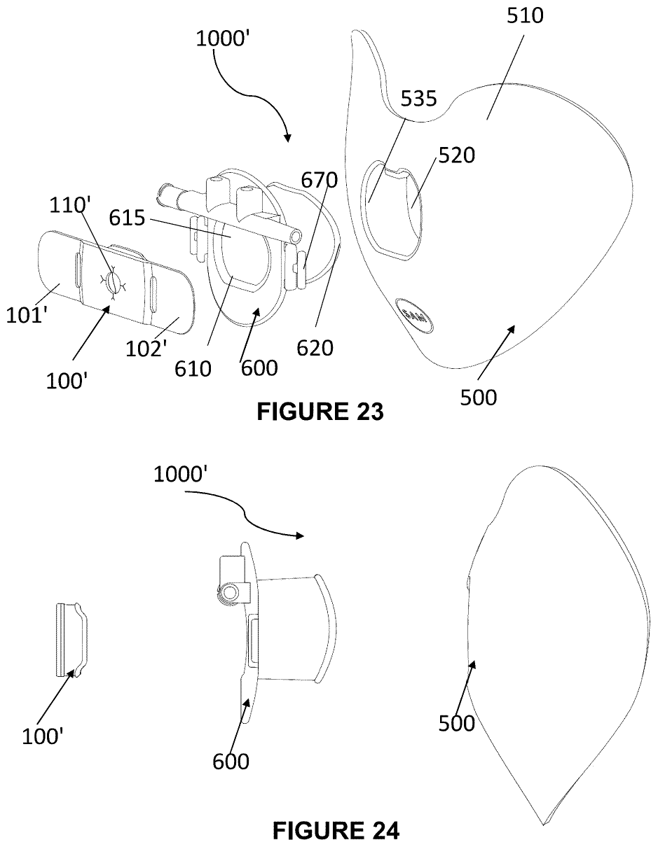

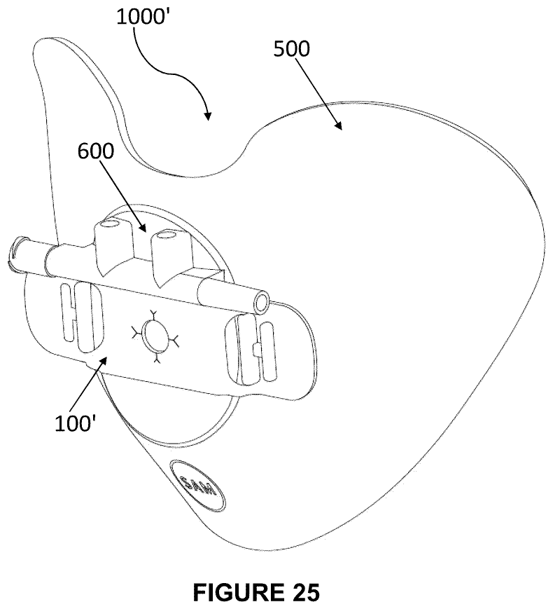

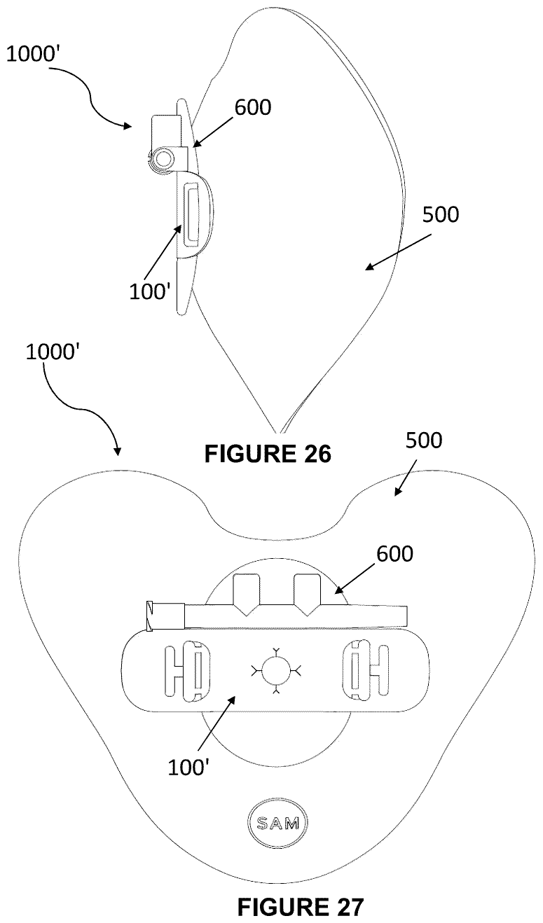

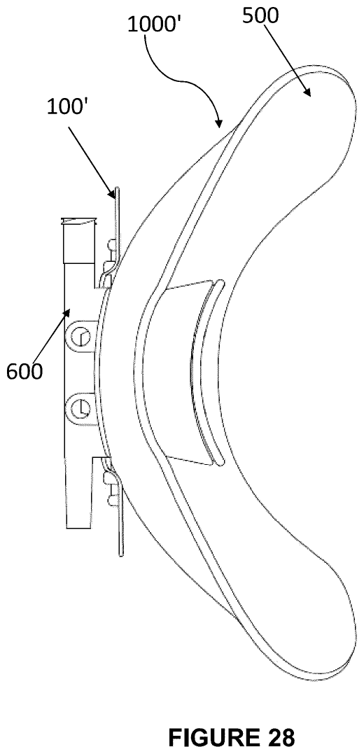

FIGS. 23 to 28 illustrate yet another embodiment of a medical apparatus 1000′ which includes another closure device 100′ which works in conjunction with the mask 500 and the bite block 600. Like reference numerals denote like features which have been previously described.

Referring to the views of the closure device 100′ in FIGS. 23 to 28, the closure device 100′ comprises a flexible member 110′ with a plurality of flexible segments defining an opening or aperture 114′. The centrally located aperture 114′ is provided in the flexible member 110′ and the width of the aperture is preferably smaller than the diameter of the endoscope tube T. As will be described in the foregoing sections, the flexible segments are adapted to flex as the endoscope tube T is pushed into the aperture 114′ for accommodating the endoscope tube T within the aperture 114′.

The closure device 100′ may includes a hollow and substantially cylindrical closure body defining a lumen that extends between a proximal end (that receives the endoscope tube T) and a distal end which, during use, is pushed into the passage 615 of the bite block 600. The flexible segments in the flexible member 110′ are radially arranged relative to the aperture 114′ to allow the size of the aperture 114′ to be increased when the endoscope tube T is pushed into the opening 114′. This configuration allows each of the flexible segments to flex when the endoscope tube T is pushed into the aperture 114′ and results in engagement between the flexible segments and the outer walls of the endoscope tube T (as shown previously in FIGS. 8 and 9) whilst also expanding the size of the aperture 114 to allow the endoscope tube T to pass through. The flexing of the flexible segments during use prevents any large gaps between the endoscope tube T and the flexible segments during use. As a result, the flexible segments function like a curtain and prevent expulsion of any droplets out of the patient's mouth during use whilst the endoscope T is in an inserted and in-use configuration.

The opening 114′ is flanked by two laterally extending fastening members 101′ and 102′. Each of these laterally extending fastening members function as attachment tabs and allow the closure member 100′ to be attached to the holding members 670 provided on the bite block 600. As a result of this feature, it may no longer be necessary to provide the substantially cylindrical closure body that is received all the way into the passage 615 of the bite block 600. Typically slits are provided along the lateral locations of the tabs 101′ and 102′ and in order to position the closure 100′ the tabs 101′ and 102′ may be stretched in a laterally outward direction to engage with the holding members 670 of the bite block 600. The provision of such an attachment mechanism also prevents the closure body 100′ form inadvertently being pushed into the passage 615 of the bite block 600.

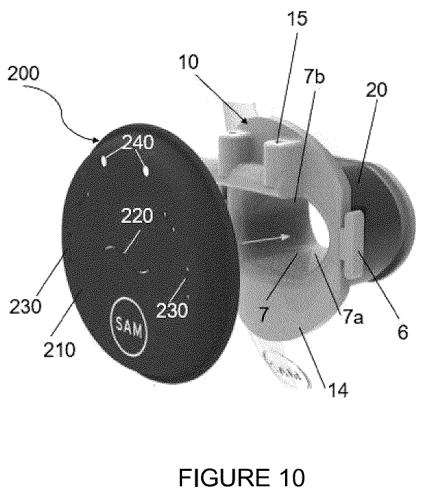

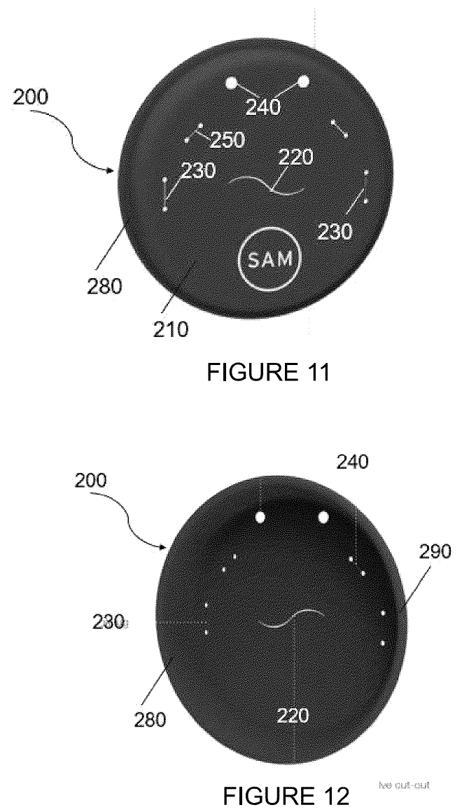

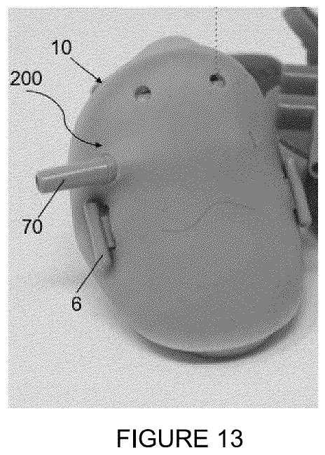

FIGS. 10 to 13 illustrate an endoscopic shield 100 for use in combination with an endoscopic mouthpiece or bite block 10 (as shown in FIGS. 10 and 13) which is similar in structure and function to the mouthpiece bite block 600 discussed in the previous sections.

The endoscopic mouthpiece 10 consists of a generally elliptical cylindrical main body 20, having a proximal end, which sits outside of a patient's mouth, and a distal end, which sits inside a patient's mouth. Main body 20 surrounds main oral passage lumen or channel 7, which is sized to allow for passage of an endoscope and ventilation of the patient. Oral passage 7 is defined by a lower surface 7 a and an upper surface 7 b. Upper surface 7 b further defines an opening for internal gas channel or lumen 15. Integral to the proximal end of main body 20 is flange 14, which sits outside of a patient's lips and serves both to locate the mouthpiece relative to the patient's mouth and protect the patient's lips and teeth from an endoscope. Flange 14 is integral to main body 20 at distal surface 22. Attached at each side of flange 14 is strap attachment wing 6 for straps that goes around the patient's head and helps secure the mouthpiece 10. It is important to note that the scope of the endoscopic shield 100 described in the foregoing sections is in no way limited to a specific type of endoscopic bite block.

The endoscopic shield 200 a flexible sheet 210 that is configured to be stretched and extended over the frontal flange 14 of the mouthpiece to cover the opening of the annular bite portion 20. The flexible sheet 210 comprises an S-shaped slit 220 for allowing insertion of an endoscope tube into the passage 7 of the annular bite portion 20 through the opening. The slit 220 is formed by flexible segments that flex for accommodating an endoscopic tube to penetrate the flexible sheet 210 and pass through into the passage 7 in an in-use engagement configuration. As the endoscope is pushed through the slit 220, the flexible segments defining the slit 220 engage with the outer walls of the endoscope (by way of movement as the tube is pushed through the slit) to effectively form a curtain that prevents expulsion of any droplets out of the patient's mouth during use. In some embodiments, two S-shaped slits may be provided in an intersecting configuration.

The slit 220 is located in between at least two spaced apart apertures 230 dimensioned to be engaged with the strap attachment members 6. During use, when the flexible sheet is in a stretched position, the apertures 130 are positioned to engage and receive the attachment wings 6 so that the slit 120 is located substantially across the opening of the annular bite portion 20 of the endoscopic mouthpiece 10. The apertures 230 are dimensioned to be smaller in size than the strap attachment member 6 to ensure that at least a part of the strap engagement member projects out of the aperture during use. The provision of the apertures 230 also helps in maintaining the flexible sheet 110 in the stretched position and provides a guide for easily aligning the flexible sheet 210 so that the S-shaped slit is appropriately positioned to guide the endoscope tube into the passage 7 of the endoscopic mouthpiece 10.

The flexible sheet 210 is also provided with a first plurality of openings 140 to correspond with exhaling ports 15 of the endoscopic mouthpiece 10 to allow exhaled gas to exit the exhaling ports 10 when the flexible sheet 210 is stretched and extended over the frontal flange 14 of the mouthpiece 10 to cover the opening of the annular bite portion 20. A second plurality of openings 150 may also be provided on the flexible sheet 210 to allow an inlet gas port 70 (See FIG. 13) of the endoscopic bite block 10 to pass through the flexible sheet and project outwardly from the endoscopic mouthpiece 10 when the flexible sheet 210 is stretched and extended over the frontal flange 14 of the mouthpiece 10 so that a gas line can be easily coupled with the inlet gas port 70.

Turning to FIGS. 12 and 13 the flexible sheet 110 includes a side wall 180 that extends towards the patient's face during use to define a hollow internal space or cavity to accommodate the frontal flange 14. A thickened return lip 290 is located along the peripheral portion of the flexible side wall 280 and the flexible side walls 280 is integrally formed with the flexible sheet 210. The return lip 290, during use, engages an edge portion of the flange 14 by lapping over the edge of the flange 14 to maintain the sheet 210 in the stretched position during use.

The provision of the endoscopic shield 200 reduces the chances of any droplets from being expelled out of the passage 7 of the mouthpiece while endoscopy is being carried. As a result, any expulsion of biological pathogens from the user's mouth via the opening of the passage 7 is reduced. The shield 200 is readily replaceable and can be mounted onto endoscopic mouthpieces of varying configurations.

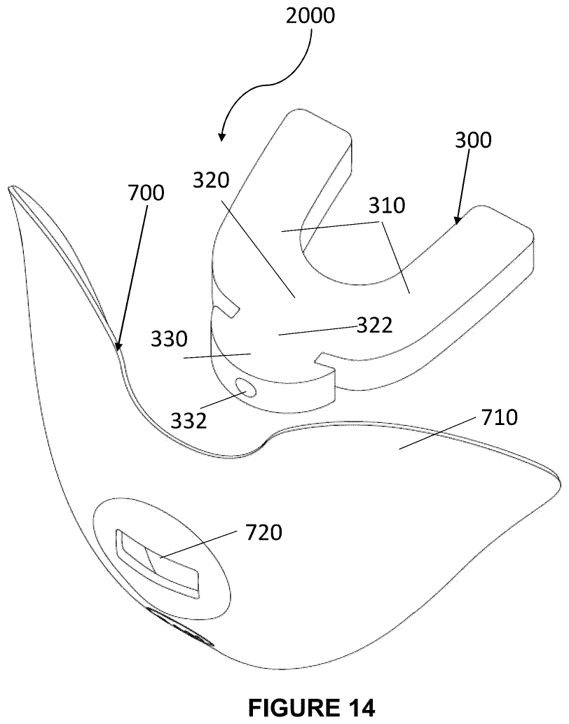

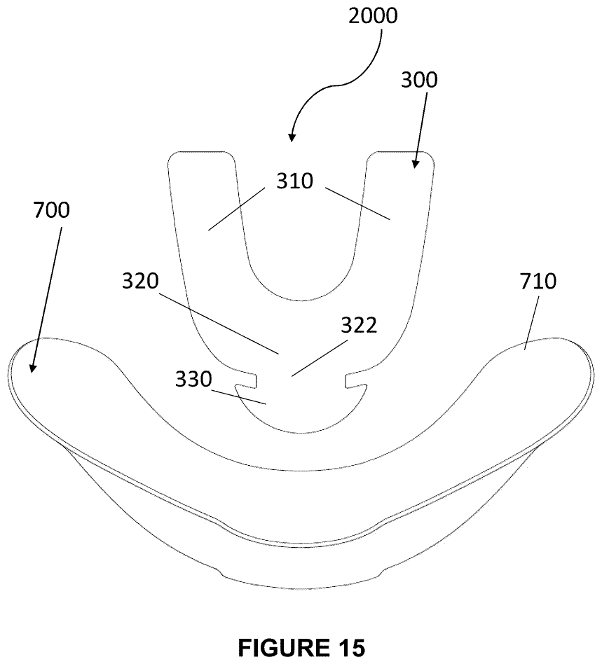

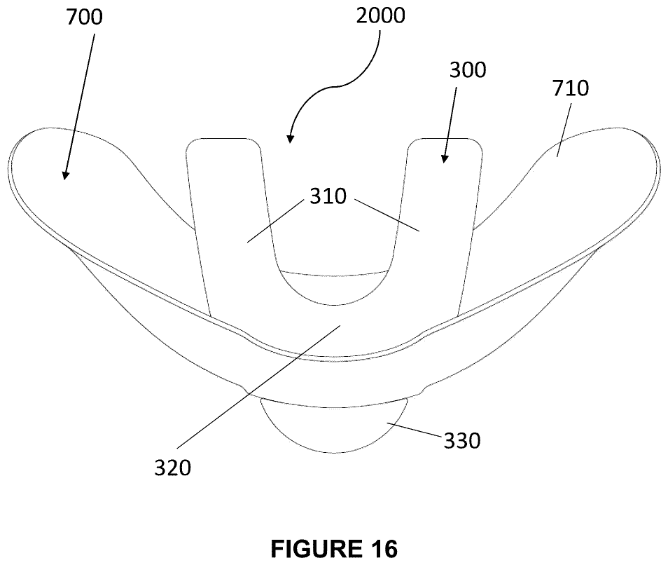

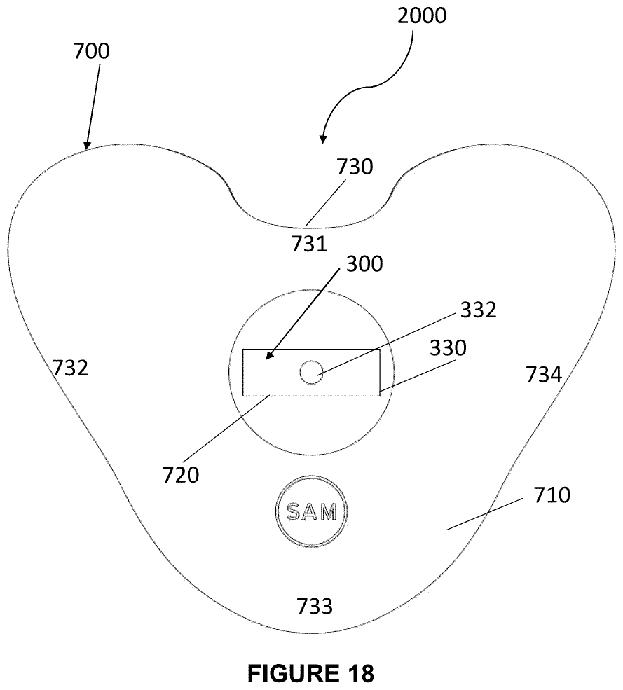

FIGS. 14 to 18 illustrate embodiments of a medical apparatus 2000 that includes a combination of an oral protector 300 and a mask 700. The oral protector 300 of the present invention is especially adapted for use in electroconvulsive therapy (ECT). However, it may be useful for other situations, including as a training device for lessening bruxism (grinding of teeth) and to facilitate the free flow of air into the oropharynx, and thence to the lungs, in individuals suffering from disorders such as sleep apnoea, in addition to its use in ECT, the oral protector 300 may be used during other medical procedures such as cardioversion/defibrillation. It may also be used during surgical and anaesthesia procedures which require oral protection or there is a risk of spontaneous seizure, for example, during a neurosurgical operation.

For the purposes of describing the medical apparatus 2000, it is important to note that the mask 700 may either be used in conjunction with the oral protector 100 described herein or may also be additionally or alternatively be used with any other prior art oral protector (not shown here). The structural configuration of the oral protector 300 is not limiting in any manner.

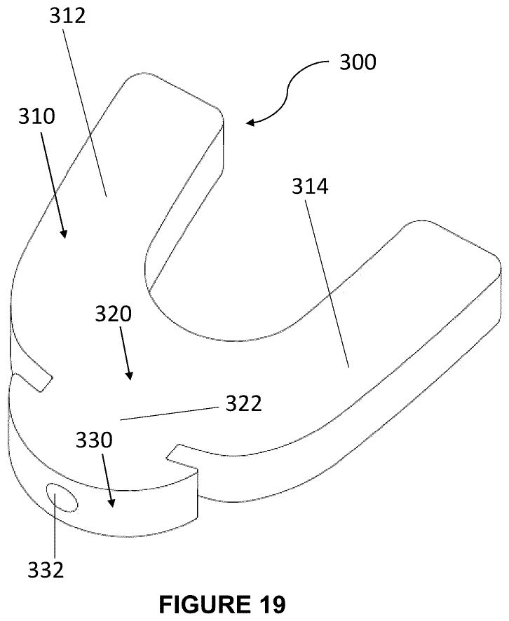

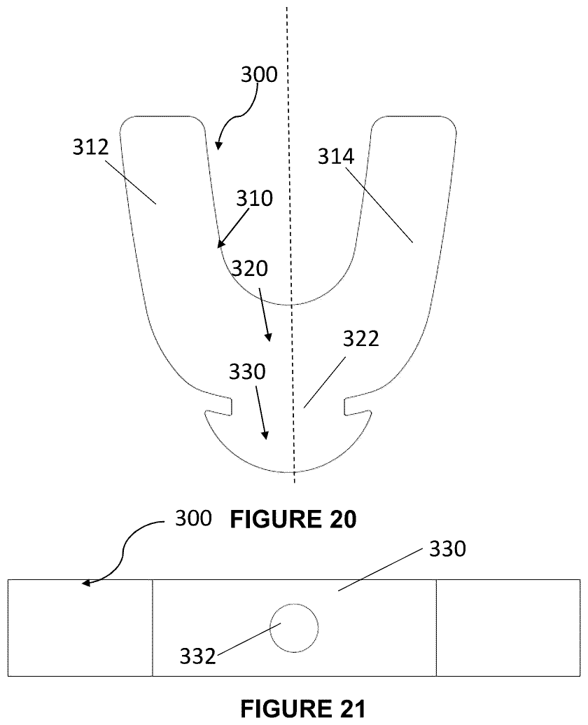

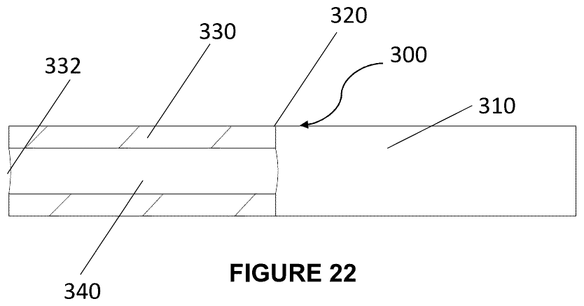

The oral protector 100 as shown in the accompanying figures (including isolated views of the oral protector shown in FIGS. 19 to 22). The oral protector 300 comprises a rear biting portion 310 with two lobes 312 and 314 separated a hollow cavity. The each of the lobes 312 and 314 for the biting portion 310 are structured for covering the molars of the patient. The two lobes 312 and 314 converge towards an anterior portion 320 of the oral protector 300 which fits between the frontal teeth (such as the canines and incisors of the patient) and the lips of the patient. The anterior portion 320 extends via a bridging portion 322 into a frontal portion 330 that is shaped to protrude out of the user's mouth during use. The frontal portion 330 comprises an opening 332 into an airway passage 340 extending through the body of the frontal portion 330 and the anterior portion 320. The passage 340 is provided to allow air or oxygen or any other medical gases into the oral cavity and eventually into the lungs of the patient when a medical procedure such as electroconvulsive therapy is being carried out.

The mask 700 as previously discussed, may be additionally used in conjunction with the oral protector 300 or any other prior art oral protector (not shown). In the following sections, the use of the mask 700 has been described by referring to the oral protector 300. However, the use of the mask 700 is not limited in this manner and the mask 700 may find use with other types of oral protectors or bite blocks not described here. The mask 700 includes a flexible sheet 710 adapted to be placed over a user's face. The mask 700 is configured to be detachably coupled with the oral protector 300 (as will be explained in further detail) and comprises a flexible sheet 710 that can be placed over the patient's face area surrounding the mouth of the patient. The flexible sheet 710 is sufficiently flexible to confirm to the contours of the patient's face. The flexible sheet 710 comprises an opening 720 that is located radially inwardly relative to the peripheral portions of the flexible sheet 710. The opening 720 is shaped and sized to allow the bridging portion 322 of the oral protector 300 to be received therein whilst preventing the frontal portion 330 of the oral protector 300 to pass through the opening 720 during use in a first operable configuration.

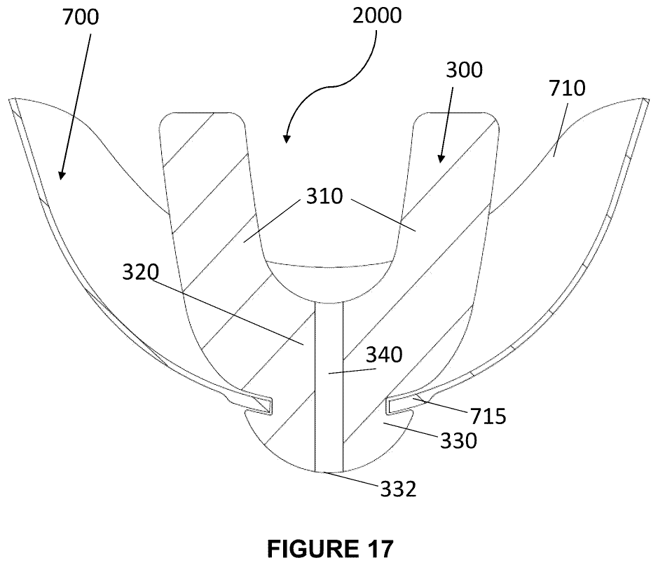

The opening 720 is sized to be narrower than the length of a rear face of the frontal portion 330 to prevent the mask 700 from becoming uncoupled from the oral protector 300. The opening 720 therefore provides an attachment structure to allow detachable attachment of the oral protector 300. The material of the flexible sheet 710 surrounding the opening 720 is sufficiently resilient to allow the opening 720 to be stretched by a user into a stretched configuration to allow the frontal portion of the oral protector to be pushed through the opening 720. Once the frontal portion 330 has been pushed through the opening 720 to extend outwardly relative to the outer surface of the flexible sheet, an outer wall of the bridging portion 322 engages a thickness of the flexible sheet 710 surrounding the opening 720. In the preferred embodiment, the portion 715 surrounding the opening 720 is slightly thicker than the remaining flexible sheet 710 as can be seen most clearly in FIG. 17. The thickened engagement portion 715 of the flexible sheet 710 helps in maintaining the attachment between the oral protector 300 and the mask 700.

The sheet 710 extends between two sides 732 and 734 with top and bottom portions 731 and 733 of the sheet 710 extending therebetween with the opening 720 being located substantially centrally relative to the peripheral portions of the sheet 710. The top portion 731 comprises a cut-out section 730 for receiving a nose of the user, the cut-out section 730 being flanked by a top left portion and a top right portion of the flexible sheet 710 to allow the cut-out section 730 to be positioned substantially above the mouth of the user when the mask 700 is coupled with the oral protector 300 during use. In other alternative embodiments, the portion of the flexible sheet 710 surrounding the opening 720 may be integrally formed with the bridging portion 322 or any other part of the anterior portion 320. In the coupled position, the flexible sheet 710 surrounds the front piece 330 of the oral protector 300 and extends over the patient's face to surround the mouth region of the patient. As a result, any droplets inadvertently released by the patient (whilst electroconvulsive therapy is being conducted) is captured along the inner walls of the flexible sheet 710 positioned in close proximity to the user's mouth. As a result, the mask 700 prevents the spread of such droplets and makes the working environment for the healthcare providers much safer and hygienic.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

What is claimed is:

1. Medical Apparatus for use in combination with an airway device to maintain a patient airway, the airway device comprising a proximal end and a distal end with a passage extending therebetween wherein the proximal end comprises an opening to receive a supply of gas for passage of air or medical gas and wherein the distal end is adapted to be inserted through the mouth of the patient, the medical apparatus comprising: a closure member configured for being inserted and retained in the passage during use wherein the closure comprises a flexible member extending across the opening, the flexible member further comprising a plurality of flexible segments being adapted to flex for accommodating an endoscopic tube to penetrate and pass through into the passage in an in-use engagement configuration wherein a portion of one or more of the flexible segments engages with outer walls of the tube to prevent expulsion of any droplets out of the patient's mouth during use;
wherein the closure member comprises a profiled stop member to limit inward movement of the closure member into the passage, the stop member comprising a circumferential flange arranged circumferentially around the flexible segments and wherein the circumferential flange has a diameter that is greater than the width of the lumen defined by outer walls of the closure body.

2. Medical apparatus in accordance with claim 1 wherein the closure member comprises a hollow closure body defining a lumen extending between a proximal end of the closure body and a distal end of the closure body with outer walls of the closure body being configured for engagement with inner walls defining the passage of the airway device.

3. Medical apparatus in accordance with claim 2 wherein the flexible member is located at or adjacent one of the two ends of the closure body.

4. Medical apparatus in accordance with claim 1 preceding comprising a plurality of said flexible segments being radially arranged relative to the opening.

5. Medical apparatus in accordance with claim 1 wherein the one or more flexible segments are attached to a radially outer portion, preferably at one end, of the closure member.

6. Medical apparatus in accordance with claim 1 comprising a plurality of flexible segments extending from a peripheral portion of the closure member in a radially inward direction towards the opening.

7. Medical apparatus in accordance with claim 1 wherein the flexible segments are separated from each other by narrow cut-out sections formed continuously with the opening to allow the flexible segments to flex and accommodate an endoscopic tube in the opening, said endoscopic tube having a diameter that is greater than the width of the opening.

8. Medical apparatus in accordance with claim 1 further comprising a flexible sheet adapted to be placed over a user's face wherein the flexible sheet comprises an opening dimensioned to receive outer walls of the closure body and prevent the proximal end of the closure body from being passed through the opening of the flexible sheet wherein the sheet is substantially impervious to air or liquids or one or more of the other medical gases.

9. (canceled)

10. Medical apparatus in accordance with claim 8 wherein width of the opening of the sheet is dimensioned to be smaller than the width/diameter of the circumferential flange of the closure member to prevent the proximal end or of the closure member from passing through the opening of the mask and becoming uncoupled from the airway device or the closure member.

11. Medical apparatus in accordance with claim 8 wherein the opening of the mask is dimensioned such that at least a portion of an underside surface of the circumferential flange of the closure member contacts a portion of the flexible sheet defining the opening to form a seal during use.

12. Medical apparatus in accordance with claim 8 wherein the flexible sheet extends between two lateral sides with top and bottom portions of the sheet extending therebetween and wherein the top portion comprises a cut-out section for receiving a nose of the user, the cut-out section being flanked by a top left portion and a top right portion of the flexible sheet.

13. Medical apparatus in accordance with claim 8 wherein the opening of the mask is located below the cut-out section for being positioned substantially above the mouth of the user.

14. Medical apparatus in accordance with claim 8 further comprising a locking member configured to be received and engaged with a locking portion located on an outer wall portion of the passage extending between the proximal end and distal end of the airway device for detachably coupling said mask with the bite block and wherein the locking member comprises a profiled projection member configured for engagement with an opening in the locking portion.

15. Medical apparatus in accordance with claim 14 wherein the locking member comprises a profiled projection member configured for engagement with an opening in the locking portion.

16. Medical apparatus in accordance with any one of claim 14 wherein during use the projection member extends in a transverse direction relative to a longitudinal axis of the passage and wherein the projection member extends in an in-use downwardly direction towards a radially central portion of the passage.

17. (canceled)

18. Medical apparatus in accordance with claim 8 further comprising a passage engaging wall portion formed integrally with said flexible sheet, the wall portion being dimensioned for surrounding and engaging an outer wall of the passage, the passage engaging wall defining the opening at an end portion of the passage engaging wall.

19. Medical apparatus in accordance with any one of claims 8 to 18 wherein at least a portion of the flexible sheet, during use, engages with a back wall of a frontal portion of the airway device.

20. Medical apparatus in accordance with any one of claim 19 when dependent upon claim 21 wherein the at least portion of the flexible sheet is integrally formed with the passage engaging wall portion.

21. A closure device for use in combination with an endoscopy bite block, the bite block including a front piece to fit over a patient's mouth which has a central opening communicating with a passage extending into the patient's mouth to receive and pass an endoscopic tube, the closure device comprising a closure member configured for being inserted and retained in the passage during use wherein the closure comprises a flexible member extending across the opening, the flexible member further comprising a plurality of flexible segments being adapted to flex for accommodating an endoscopic tube to penetrate and pass through into the passage in an in-use engagement configuration wherein a portion of one or more of the flexible segments engages with outer walls of the tube to prevent expulsion of any droplets out of the patient's mouth during use;
wherein the closure member comprises a profiled stop member to limit inward movement of the closure member into the passage, the stop member comprising a circumferential flange arranged circumferentially around the flexible segments and wherein the circumferential flange has a diameter that is greater than the width of the lumen defined by outer walls of the closure body.

22. Medical apparatus for use in combination with an endoscopic mouthpiece, the endoscopic mouthpiece comprising: an annular bite portion for placement within the mouth of a patient so as to keep the mouth of the patient open and provide an open passage into the mouth of the patient with an opening to insert an endoscope therethrough; a frontal flange extending radially outwardly at one end of said annular bite portion for placement outside the mouth of the patient to overlap the patient's facial area surrounding the mouth of the patient; strap attachment members projecting upwardly from said flange; and one or more ports provided on said frontal flange for delivering gas to the patient for breathing; wherein the medical apparatus comprises a flexible sheet configured to be stretched and extended over the frontal flange of the mouthpiece to cover the opening of the annular bite portion, the flexible sheet comprising one or more slits for allowing insertion of an endoscope tube into the passage of the annular bite portion through the opening wherein the slit is formed by flexible segments being adapted to flex for accommodating the endoscopic tube to penetrate the flexible sheet and pass through into the passage in an in-use engagement configuration wherein a portion of one or more of the flexible segments engages with outer walls of the tube to prevent expulsion of any droplets out of the patient's mouth during use.

23-57. (canceled)
